(19) United States
(12) Patent Application Publication
(10) Pub. No.: US 2021/0252030 A1
(43) Pub. Date: Aug. 19, 2021

(54) NOVEL DOSAGE FORMS OF LACTULOSE

(71) Applicant: P. SIVARAJAKUMAR, Reddiyarpalayam Pondicherry (IN)

(72) Inventor: P. SIVARAJAKUMAR, Reddiyarpalayam Pondicherry (IN)

(21) Appl. No.: 17/251,429

(22) PCT Filed: Oct. 16, 2018
(86) PCT No.: PCT/IN2018/000047

§ 371 Date: Dec. 11, 2020

(30) Foreign Application Priority Data
Jun. 13, 2018 (IN) 201841022062

(51) Int. Cl. A61K 31/7016; A61K 9/00; A61K 47/12; A61K 47/26; A61K 47/10; A61K 47/44; A61K 45/06

(52) U.S. Cl. CPC A61K 31/7016; A61K 9/0056; A61K 47/12; A61K 45/06; A61K 47/10; A61K 47/44; A61K 47/26

(57) ABSTRACT

The present invention discloses oral pharmaceutical compositions of Lactulose particularly useful to treat Constipation and hepatic encephalopathy. In particular, the present invention provides consumable, novel gummy delivery systems comprises Lactulose and methods of making the same. The composition comprises Lactulose, gelling agent, sweetener, buffering agents, flavouring agent, colouring agent, anti-tacking agent and preservative. Such gummy compositions for Lactulose may provide improved patient compliance relative to non-gummy compositions. Further the composition comprises Vitamin, nutritional supplements, minerals, antioxidants, soluble and insoluble fibre, herbs, plants, Probiotics, Pre-biotics amino acids, and digestive enzymes.

### FIELD OF THE INVENTION

The present invention discloses consumable, novel gummy delivery systems comprise Lactulose and methods of making the same. Further the composition comprises Vitamin, nutritional supplements, minerals, antioxidants, soluble and insoluble fibre, herbs, plants, amino acids, Probiotics, Pre-biotics and digestive enzymes.

### BACKGROUND OF THE INVENTION

Constipation is a medical condition which causes people to have difficulty getting rid of solid waste from their body. Constipation is one of the most prevalent gastrointestinal complaints and annually, more than 2.5 million physician visits are for constipation and more than $500 million is expended for prescription and non-prescription laxatives. Low fibre intake, inadequate hydration, reduced mobility as the result of general functional decline and institutionalization, reduced sensation of thirst, electrolyte disturbances (hypercalcemia, hypokalemia, hypermagnesemia), endocrine and metabolic disorders (eg, diabetes mellitus, hyperparathyroidism, hypothyroidism, chronic renal failure), neurological disorders (eg, dementia, Parkinson disease, neuropathies, multiple sclerosis, spinal cord injuries, cauda equine syndrome), psychological comorbidities (eg, depression, distress, personality disorders, or history of abuse), and concurrent medications (eg, anticholinergics, diuretics, β-blockers, opiates, iron supplements, calcium channel blockers, antidepressants, acetaminophen, aspirin and NSAIDs) all are said to contribute to chronic constipation, especially in the elderly.

Lactulose is a nondigestible disaccharide (sugar) synthesized from fructose and galactose and is used to treat constipation and some liver diseases. Lactulose passes unabsorbed down to the large intestine where resident bacteria consume it and produce lactic, acetic, and formic acids, which draw fluid into the bowel to soften the stool (laxative effect). Acidification of the colon contents attracts ammonia from the bloodstream, assisting stool excretion; helpful in liver failure when ammonia cannot be detoxified.

Lactulose, β-galactosido-fructose, is a synthetic disaccharide which is not digested in the small intestine since the specific disaccharidase is lacking. It passes unchanged into the colon where it serves as an energy source for the carbohydrate-splitting bacteria, predominantly Lactobacillus acidophilus and L. Bifidus. During the fermentation process, low molecular organic acids (mainly formic and lactic) are formed which in turn lowers the pH of stool.

Numerous delivery systems are available in the market for delivery of active ingredients. Generally, most children, elder populations and patients with dysphagia cannot swallow traditional solid dosage forms (e.g., tablets and capsules) at least due to the risk of choking.

For young children (i.e., <2 years of age), liquid dosage forms are preferred as dosing can be facilitated via an oral syringe or spoon. These dosage forms, however, can be problematic as they are not very attractful for children to take the product due to characteristics odour and taste. Suspensions can improve taste-masking effectiveness, however, mouth feel and grittiness is often the overriding issue.

Patient compliance is a problem with current commercially available dosage forms. For example, problems with compliance are sometimes seen when pregnant women have a condition that does not allow them to easily take current commercially available dosage forms, including morning sickness or nausea and vomiting of pregnancy.

Accordingly, a need exists for compositions that provide suitable nutritional supplementation and that have satisfactory patient compliance. Such compositions may be used, for example, before and during pregnancy.

It is therefore, an object of the present invention to provide new delivery systems for actives which are gummy and which overcome of the drawbacks of delivery systems currently available.

Gummy dosage forms are particularly effective for enabling compliant dosing in children, elder populations, patients with dysphagia and pregnant women as they provide a palatable, chewable base and can incorporate active ingredient(s) that are generally of very low dose, have the ability to withstand the high thermal stress of the gummy manufacturing process, and have low intrinsic taste response. Moreover, while gummy dosage forms provide the basis for effective dosing of active ingredients to children, their application for the delivery of Food supplements and like materials has been highly restrictive due to the limited number of active ingredients that are compatible with the gummy dosage-platform.

Gummy dosage forms have previously been produced by compounding a variety of ingredients (e.g., sugars, corn syrup, water, flavours, and other sweeteners) then cooking the mixture at high temperatures (e.g., up to about 240° C.) before depositing the cooked mixture into preformed moulds. The incorporation of the active ingredients can be facilitated only during the initial compounding step prior to cooking. The viscosity of the cooked mixture is generally too high to enable the active ingredients to be added retrospectively. As a result of the very high thermal stress of the cooking process, the active ingredients can be subject to significant chemical and/or physical degradation during the manufacture of gummies. In the present invention, the gummies were obtained at about 80° C. using Pectin, Carrageenan, combination of Pectin and Carrageenan as gelling agent. Accordingly, the practice of utilizing overages (including excess active ingredient to off-set the losses due to degradation during manufacturing) has been instituted.

The use of overages to off-set gross manufacturing losses in gummy dosage forms is permitted only for some functional actives that do not present safety concerns. The application of this practice for Food supplements is not generally feasible as it may lead to significant efficacy, safety, and regulatory issues. In addition, as the quality control requirements for Food supplements (i.e., claimed dose of active, content uniformity, degradation limits, etc.) are generally much more stringent than food-based functional additives, the suitability of gummies as an oral delivery platform becomes even more prohibitive. As such, there remains a need in the art for oral, chewable dosage forms suitable for delivery of APIs and the like in a manner where active ingredient content can be closely controlled throughout manufacturing to provide a resulting dosage form of consistent quality and desirable palatability.

The present invention provides novel consumable gummy composition comprising Lactulose that are easily administered to an infant or adult. When taken directly by mouth or added to food or infant formula, the composition has a desirably bland flavour and is easy to consume. Moreover, the high density of Lactulose per unit volume of the composition results in a minimal amount of dosage to be administered. This makes the composition relatively innocuous and easy for infants to consume.

Compositions such as Gummies can be made with a ‘fizzy’ effect which stimulates saliva and makes the experience more enjoyable. These compositions also provide a way of converting poorly soluble APIs into a user-friendly form. For people with dysphagia (difficulty swallowing), these compositions are an excellent alternative to conventional tablets. In addition, these compositions reduce the risk of drug-induced esophagitis—when a tablet is caught in the esophagus and dissolves while remaining in contact with the sensitive esophagus lining.

Gummy dosage form results with more rapid drug absorption from the pre-gastric area i.e. mouth, pharynx and oesophagus which may produce rapid onset of action in cases such as motion sickness, sudden episodes of allergic attack or coughing, where an ultra-rapid onset of action required. This dosage form allow high drug loading and have sufficient strength to withstand the rigors of the manufacturing process and post manufacturing handling. Pre-gastric absorption from gummy dosage form can result in improved bioavailability, reduced dose and improved clinical performance by reducing side effects. Pregastric drug absorption avoids the first-pass metabolism; the drug dose can be reduced if a significant amount of the drug is lost through the hepatic metabolism.

Gummies contain medication in a flavoured gummy candy and Good for children and elder patients. Gummies are generally pectin, Carageenan or combination of pectin and Carageenan based dosage forms and can be considered as lozenges and troches. The dosage form has become exceedingly popular. The forms generally contain natural or artificial sweeteners, natural or artificial colours, acidulents, flavours and anti-sticking agents and texture improving agents.

Patient compliance and safety are generally at the top of the list of healthcare practitioners when recommending pharmaceutical dosage forms. Convenience is also often high on the list when choosing dosage forms by a consumer. With these thoughts being considered, the use of novel compositions such as mouth melt granules, Dry/wet suspension and Gummies could assist in improving compliance in children and elder patients.

### SUMMARY OF THE INVENTION

In one general aspect, there is an oral pharmaceutical compositions comprising Lactulose and one or more pharmaceutically acceptable excipients.

The present invention provides consumable, novel gummy delivery systems comprises Lactulose and methods of making the same.

In another aspect, the present invention discloses consumable, novel gummy delivery systems comprising Lactulose and one or more pharmaceutically acceptable excipients.

In another aspect, gummies of Lactulose further comprises one or more excipients selected from the group consisting of Binders, Glidants, Buffers, stabilizing agents, chelating agents, solubilizing agents, processing aids, lubricating agents, preservatives, opaquing agents, colorants, sweetening agents, lubricants, coating agents, glazing agents, flavouring agents and diluents.

The present invention provides consumable, novel gummy delivery systems comprises Lactulose, Vitamin, nutritional supplements, minerals, antioxidants, soluble and insoluble fibre, herbs, plants, amino acids, Pre-biotic, Probiotic and digestive enzymes.

### DETAILED DESCRIPTION OF THE INVENTION

The term “gummies” is intended to include chewing gum, troches, candy, lozenges and all shapes of gummies dosage forms.

In some embodiments, consumable, novel gummy delivery systems comprises Lactulose and one or more inactive ingredients that include but are not limited to ethanol, water, propylene glycol, buffers (including, by way of example and without limitation, phosphate buffers, citrate buffers, lactic acid, Tris buffer, TPE buffer, sodium bisulfate, sodium citrate and others known to those of ordinary skill in the art), stabilizing agents (including, by way of example and without limitation, antioxidants (e.g., ascorbic acid, propionic acid, sodium bisulfite, sodium sulfite, vitamin E, i e tocopherol, sodium pyrosulfite, butylhydroxytoluene, butylated hydroxy anisole, edetic acid and the like), chelating agents (e.g., fumaric acid, sodium edetate, and the like), and others known to those of ordinary skill in the art), antifoaming agents (e.g. sorbitan trioleate, etc.), detergents (e.g. sucrose stearate, etc.), solubilizing agents (e.g. polyethylene glycol 400 monostearate, etc.), and others known to those of ordinary skill in the art), processing aids (e.g. substances used to assist processing, including, by way of example and without limitation, lubricating agents, antioxidants, and others known to those of ordinary skill in the art), emulsifiers (including, by way of example and without limitation, synthetic (e.g. sodium lauryl sulfate, potassium laurate, etc.), natural (e.g. lecithin, etc.), and finely divided solid emulsifiers (e.g. bentonite, magnesium hydroxide, etc.), and others known to those of ordinary skill in the art), suspending agents (including, by way of example and without limitation, cellulose derivatives (e.g. carboxymethylcellulose, methylcellulose, ethyl cellulose, etc.), natural polymers (e.g. alginates, xanthan gum, guar gum, etc.), synthetic polymers (e.g. carbomers, polyvinyl pyrrolidone, etc.), clays (e.g. magnesium aluminum silicate, hectorite, etc.), and others known to those of ordinary skill in the art), preservatives (including, by way of example and without limitation, citric acid, tartaric acid, lactic acid, malic acid, acetic acid, benzoic acid, and sorbic acid, benzethonium chloride, benzyl alcohol, cetrimide, glycerin, propylene glycol, benzoic acid and sodium benzoate, potassium sorbate, Sodium Benzoate and sorbic acid, and others known to those of ordinary skill in the art), opaquing agents (including, by way of example and without limitation, titanium dioxide, and others known to those of ordinary skill in the art), colorants (including, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, sunset yellow supra, Tartrazine Yellow, Quinoline yellow Supra, Quinoline yellow Supra, Brilliant blue supra, caramel, ferric oxide, red, pigments, dyes, tints, titanium dioxide, natural colouring agents, such as grape skin extract, beet red powder, beta carotene, annato, carmine, turmeric, paprika, black carrot juice, and others known to those of ordinary skill in the art), sweeteners or sweetening agents (including, by way of example and without limitation, sucrose, fructose, fructose, high fructose corn syrup, dextrose, saccharin sodium, maltodextrin, aspartame, potassium acesulfame, neohesperidindihydrochalcone, sucralose, monoammonium glycyrrhizinate, and others known to those of ordinary skill in the art), lubricants, such as calcium stearate, glycerylbehenate, magnesium stearate, mineral oil, polyethylene glycol, sodium stearyl fumarate, stearic acid, talc, vegetal oil, sodium lauryl sulfate or zinc stearate; coating agents, such as castor oil and sorbitol; perfuming agents (including, by way of example and without limitation, natural flavor oil, a synthetic flavor oil, and others known to those of ordinary skill in the art), glazing agents (including, by way of example and without limitation, vegetable oil, beeswax, carnauba wax, and others known to those of ordinary skill in the art), disintegrants (including alginic acid, croscarmellose sodium, crospovidone, potassium polacrilin, sodium starch glycolate, salts of carboxymethylcellulose and starch). Additional examples of other inactive ingredients are well known in the art. See, e.g., Remington: The Science and Practice of Pharmacy (21st ed. 2005).

As stated above, the gelling compound or binding agent may include pectin, food starch, Carrageenan, gum, or any other suitable binder, or combination thereof. For example, the binding agent may include gelling compounds including pectin products may include high (methyl) ester or low (methyl) ester pectin products made from fruit sources, such as apples, apricots, carrots, citrus fruits, or any other suitable pectin product. Such products may include, for example, UNIPECTIN® HM-pectin and/or UNIPECTIN® LM-pectin products.

Examples of gelling compounds including starch ingredients may include corn starch, rice starch, potato starch, starch derivatives, and the like. They can be used as Anti-tacking and thickening agent.

Examples of gelling compounds including Carrageenan ingredients may include kappa (K) carrageenan sold under the Gelcarin® brand, or lambda (λ) carrageenans sold under the Viscarin® brand, both available from FMC Corporation and from any other source.

The gelling agent is an essential ingredient for gummies composition and will suitably form 0.5-10% w/w of the composition, preferably 0.5-6% w/w and most preferably 1-2% w/w of the composition either alone or in combination with multiple gelling agent.

The buffer is an essential ingredient for gummies composition and will suitably form 0.5-10% w/w of the composition, preferably 0.5-2.5% w/w and most preferably 0.5-1% w/w of the composition.

The stabilizing agents for gummies composition will suitably form 0.1-2% w/w of the composition, preferably 0.5-1.5% w/w and most preferably 0.1-0.5% w/w of the composition.

The chelating agents for gummies composition and will suitably form 0.5-2.5% w/w of the composition, preferably 1-2% w/w and most preferably 0.5-1% w/w of the composition.

The anti-foaming agents for gummies composition and will suitably form 0.5-2% w/w of the composition, preferably 1-1.5% w/w and most preferably 0.5-1% w/w of the composition.

The solubilizing agents are essential ingredients for gummies. The solubilizing agents will suitably form 5-10% w/w of the composition, preferably 1-5% w/w and most preferably 02-4% w/w of the composition.

The emulsifiers are essential ingredients for gummies. The emulsifiers will suitably form 1-5% w/w of the composition, preferably 2-4% w/w and most preferably 1-2% w/w of the composition.

The preservatives are essential ingredients for gummies and will suitably form 1-4% w/w of the composition, preferably 2-3% w/w and most preferably 1-1.5% w/w of the composition.

The opaquing agents are essential ingredients for gummies. The opaquing agents will suitably form 1-2% w/w of the composition, preferably 0.5-1% w/w and most preferably 0.1-0.5% w/w of the composition.

The colorants are essential ingredients for gummies. The colorants will suitably form 1-2% w/w of the composition, preferably 0.01-1% w/w and most preferably 0.1-0.5% w/w of the composition.

The lubricant is essential ingredient for gummies composition. The lubricant will suitably form 0.1-5% w/w of the composition, preferably 1-2.5% w/w and most preferably 0.5-2% w/w of the composition.

In certain specific embodiments, gummy compositions for Lactulose supplementation disclosed herein may comprise a flavorant. In some embodiments, the flavorant may be citrus oil, a fruit essence, an extract from a plant, an extract from a leaf, an extract from a flower, an extract from a fruit, or another masking flavour known to those of ordinary skill in the art. In some embodiments, the flavorant may be one or more of anise oil, cinnamon oil, peppermint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, grape oil, apple essence, pear essence, peach essence, berry essence, wild berry essence, date essence, blueberry essence, kiwi essence, strawberry essence, raspberry essence, cherry essence, plum essence, pineapple essence, and apricot essence. Additionally, the flavorant may be one or more selected from the group consisting of citric acid, malic acid, vanilla, vanillin, cocoa, chocolate, and menthol. In some embodiments, the flavorant may include one or more of a natural plum flavor, natural apple flavor, natural mixed berry flavor, Katcha Mango Flavour, and natural cherry flavor. In some embodiments, the gummy compositions disclosed herein may comprise a masking flavor. In some embodiments, the one or more flavorants of the gummy composition may mask the taste of iron and/or DHA. In some embodiments, the one or more flavorants may increase patient compliance.

The flavouring agents are essential ingredients for gummies and will suitably form 0.001 to 0.5% w/w of the composition, preferably 0.001 to 0.5% w/w and most preferably 0.001 to 0.5% w/w of the composition.

Gummy formulations can be prepared by reported methods which are known to a person skilled in the art. Homogenous mixture of elastic, continuous glycerylated gelling agent matrix which can be readily soluble in aqueous media is formed with one or more active ingredients. A glycerylated gelling agent matrix may be prepared by heating an aqueous solution of gelling agent and glycerin to a temperature and for a time sufficient to remove from about 10% to about 80% of the initial moisture content of the aqueous solution.

Sweeteners are added essentially to improve the taste. Sweeteners comprise one or more synthetic or natural sugars, i e any form of carbohydrates suitable for use as sweetener, as well as so called artificial sweeteners such as saccharin, sodium saccharin, aspartame, e g NutraSweet®, acesulfame or Acesulfame K, potassium acesulfame, thaumatin, glycyrrhizin, sucralose, dihydrochalcone, alitame, miraculin, moneHin, stevside, neotame, sugar alcohols, such as sorbitol, xylitol, single sugars including sugars extracted from sugar cane and sugar beet (sucrose), dextrose (also called glucose), fructose (also called leavulose), and lactose (also called milk sugar); sorbitol, mannitol, glycerol, xylitol, erythritol, maltitol syrup (or hydrogenated starch hydrolyzate), isomalt, lactitol; and mixtures of sugars including glucose syrup, e g starch hydrolysates, containing a mixture of dextrose, liquid glucose, maltose and a range of complex sugars, invert sugar syrup, e g sucrose inverted by invertase (also called sucrase or sacchrase) containing a mixture of dextrose and fructose, high sugar content syrups such as treacle and honey containing a mixture of particular leavulose, dextrose, maltose, lactitole, sucrose, resins, dextrin and higher sugars; and malt or malt extracts.

The sweeteners are essential ingredients for gummies and will suitably form 1-90% w/w of the composition, preferably 5-90% w/w and most preferably 5-15% w/w of the composition.

In the framework of this invention, binders refer to excipients which enhance the linkage between particles. They include in a non-limiting manner, any of acacia, alginic acid, carbomer, sodium carboxymethylcellulose, dextrin, ethylcellulose, glucose, guar gum, hydroxypropylcellulose, maltose, methylcellulose, povidone, polyvinylpyrrolidone, starch, methylcellulose or polyethylene oxide, starch, sugars such as sucrose, glucose, dextrose, and lactose, natural and synthetic gums, carboxymethylcellulose, methylcellulose, polyvinylpyrrolidone, ethylcellulose and waxes.

Glidants may be useful in the early stages of the process of the invention in order to improve the flow ability of the powder/granules. Suitable glidants within the scope of the invention are, in a non-limiting manner, silicon dioxide, colloidal or fumed silica, magnesium stearate, calcium stearate, stearic acid, corn starch, talc, colloidal silicon, magnesium trisilicate, starch, talc or tribasic calcium phosphate, sugar alcohols, sorbitol, xylitol, mannitol, isomalt, maltitol, inositol, lactol or mixtures of two or more of these sugar alcohols;

The coating agent can be made from any commercially available powder mix for preparing coating suspensions. Examples of such powders or mix are, Opagloss® which comprises bees wax, Carnauba wax and Mineral Oil.

In some embodiments, the gummies dosage form disclosed herein may be packaged as kits using materials known to those of ordinary skill in the art. In some embodiments, the kit may be packaged in a bottle, sachet or package. In such embodiments, a kit may comprise one or more individual dosage forms. In some embodiments, each kit may comprise two individual dosage forms. In some embodiments, each kit may comprise three individual dosage forms. In some embodiments, a kit may comprise a total dosage form.

Another embodiment of the invention may comprise kits comprising gummy compositions packaged in Pillow packs. Pillow pack as packaging for gummy compositions is well known to those of ordinary skill in the art. Pillow packs may be made of a transparent plastic sheet which has been formed to carry gummies. A foil or plastic backing is then adhered across the plane of the sheet sealing the gummy compositions in their respective pouch. Examples of materials used for the pillow pack include, but are not limited to, aluminium, paper, polyester, PVC, and polypropylene. Alternative materials are known to those of ordinary skill in the art. To remove a gummy composition, the pack is peeled off and the composition is taken through the backing material.

### Manufacturing Delivery System

Lactulose solution is heated in steam jacket vessel at 80° C. Liquid glucose and Sugar or Sorbitol powder were mixed well until the solids are dissolved completely to get clear solution. Glycerin or pectin or combination of Glycerin and Pectin, Sugar or Sorbitol, Trisodium citrate, Citric acid in polybag was weighed. The mixed content was soaked in purified water (containing dissolved Potassium sorbate and Sodium benzoate) and stirred well in clean SS Vessel. The soaked content was poured in Steam jacket and mix well for about 20 minutes at 80° C. The resultant mixture is mixed in cooking tank, maintain the temperature 80° C. and transfer the content into mixing tank. Colorant was dissolved and adjusted the pH using 50% Citric acid solution if required and finally add the Flavour and mix well. The viscous suspension was transferred from mixing tank to deposition unit and maintained the temperature at 65° C. while deposition of mixed content in moulds which is prelubricated with Opaglos-Olive oil mixture Spray. The resultant Gummy gel was passed through cooling tunnel. Gummies were collected in trays and check the average weight before drying. Gummies were dried at 45° C.±5° C. in tray kept at Hot air Oven until the water content reaches between 4 to 5% w/w. The mixture of Opagloss and Olive oil was sprayed in a coating pan containing the dried gummies. The dried gummies were finally packed in the bottle pack.

1. (canceled)

2. (canceled)

3. The pharmaceutical composition of claim 6 wherein said oral pharmaceutical compositions further comprises Vitamin, nutritional supplements, minerals, antioxidants, soluble and insoluble fibre, herbs, plants, amino acids, Probiotic, Prebiotic and digestive enzymes.

4. The pharmaceutical composition of claim 6 wherein said gummies of Lactulose further comprises one or more excipients selected from the group consisting of Binders, Glidants, Buffers, stabilizing agents, chelating agents, solubilizing agents, processing aids, lubricating agents, preservatives, opaquing agents, colorants, sweetening agents, lubricants, coating agents, glazing agents, flavouring agents and diluents.

5. (canceled)

6. A gummy dosage form comprising:
lactulose and one or more pharmaceutically acceptable excipients;
wherein lactulose in the dosage form is 50 mg-12 gm,
the pharmaceuticals acceptable excipients comprises of gelling agent in a concentration range of 0.5-10% w/w of the composition, preferably 0.5-6% w/w and most preferably 1-2% w/w of the composition either alone or in combination with multiple gelling agent, and other additives, and
wherein the gelling agents comprises of Pectin, Carrageenan and other gelling agents derived from sources other than animals.

7. The gummy dosage form as claimed in claim 6, wherein the other additives comprises buffer—5-10% w/w, stabilizing agents—0.1-0.5% w/w, chelating agents—0.5-2.5% w/w, anti-foaming agents—0.5-2% w/w, solubilizing agents—0.5-10% w/w, emulsifiers—1-5% w/w, preservatives—1-4% w/w , opaquing agents—1-2% w/w, colorants—0.1-2% w/w, lubricant—0.1-5% w/w, and flavouring agents—0.001 to 0.5% w/w.

8. A process of preparation of a gummy formulation of Lactulose, comprising of steps:
preparation of Lactulose concentrate (50 mg-12 g by weight);
preparation of sweetener solution;
weighing of preservative, Glycerin or gelling agent or combination of Glycerin and gelling agent, Sugar or Sorbitol, pH adjusting agent, Trisodium citrate, Citric acid;
dissolution of preservative, potassium sorbate and sodium benzoate in purified water and stirred well in clean SS Vessel;
pouring of the potassium sorbate and sodium benzoate solution in steam jacket and mixing for 20 minutes at 80° C. to yield a mixture;
stirring the mixture and mixing in cooking tank, maintain the temperature 80° C.;
transferring of the content into mixing tank;
dissolving the colorant and adjusting the pH with pH adjusting agent and mixing flavor to yield a viscous suspension;
transferring of the viscous suspension from mixing tank to deposition unit and maintaining the temperature at 65° C. resulting in gummy gel;
passing the gummy gel through cooling tunnel;
ejection of the gummy formulation and drying at 45° C.±5° C. tray in Hot air Oven to yield final water content in gummies between 4 to 5% w/w;
lubrication of gummies spraying of opagloss and olive oil on the dried gummies and packing,
wherein the sweetener comprises of liquid glucose, sugar, or sorbitol solution, pH adjusting agent comprises of citric acid, and gelling agent is selected from pectin, carrageenan, or any other gelling agent from non-animal source.
